(19) United States
(12) Patent Application Publication
(10) Pub. No.: US 2021/0311295 A1
(43) Pub. Date: Oct. 7, 2021

(54) INFORMATION PROCESSING APPARATUS, INFORMATION PROCESSING METHOD, AND OPERATION MICROSCOPE APPARATUS

(71) Applicant: Sony Group Corporation, Tokyo (JP)

(72) Inventors: Tomoyuki OOTSUKI, Kanagawa (JP); Tatsumi SAKAGUCHI, Kanagawa (JP); Yoshitomo TAKAHASHI, Kanagawa (JP)

(73) Assignee: Sony Group Corporation, Tokyo (JP)

(21) Appl. No.: 17/349,926

(22) Filed: Jun. 17, 2021

Related U.S. Application Data
(62) Division of application No. 15/504,980, filed on Feb. 17, 2017, now abandoned.

(30) Foreign Application Priority Data
Oct. 3, 2014 (JP) 2014-205279

(51) Int. Cl. G02B 21/36; G01B 9/02; A61B 3/10; A61B 3/13; A61B 90/20; A61B 5/00

(52) U.S. Cl. CPC G02B 21/367; G01B 9/02091; A61B 3/102; A61B 3/13; A61F 9/007; A61B 90/20; A61B 5/0066; A61B 5/6821; G01B 9/02087; A61B 3/132

(57) ABSTRACT

A surgical image processing apparatus, including circuitry that is configured to perform image recognition on an intraoperative image of an eye. The circuitry is further configured to determine a cross-section for acquiring a tomographic image based on a result of the image recognition.

### CROSS-REFERENCE TO RELATED APPLICATIONS

This application is a Divisional of U.S. application Ser. No. 15/504,980, filed Feb. 17, 2017, which is based on PCT filing PCT/JP2015/004693, filed Sep. 15, 2015, and claims priority to Japanese Priority Patent Application JP 2014-205279 filed Oct. 3, 2014, the entire contents of each are incorporated herein by reference.

### TECHNICAL FIELD

The present technique relates to an information processing apparatus, an information processing method, and an operation microscope apparatus that are used for guiding an operation on an eye.

### BACKGROUND ART

In recent years, in operations on eyes, an operation guide apparatus is being used. The operation guide apparatus generates guide information to be an operation guide based on image information of an eye as an operation target and presents it to a user. The user can perform an operation while referencing the guide information, with the result that a user's lack of experience can be compensated for or an operation error can be prevented from occurring. In addition, it helps improve an operation accuracy.

As the operation guide information, there is a tomographic image obtained by an OCT (Optical Coherence Tomography). The OCT is a technique of irradiating infrared rays onto an operation target eye and restructuring reflected waves from tissues of the eye to generate an image, and a tomographic image of an eye regarding a specific cross-section is obtained. For example, Patent Literature 1 discloses an ophthalmological analysis device that presents a tomographic image of an eye obtained by the OCT to a user.

### CITATION LIST

### Patent Literature

### [PTL 1]

### Japanese Patent Application Laid-open No. 2014-140490

### SUMMARY

### Technical Problem

When acquiring a tomographic image by the OCT, a cross-section thereof needs to be designated. However, it is difficult to readily designate an optimal cross-section as the operation guide information due to the reasons that the cross-section that an operator wishes to reference changes dynamically, an eyeball moves even during an operation, and the like.

In view of the circumstances as described above, the present technique aims at providing a surgical image processing apparatus, an information processing method, and a surgical microscope system that are capable of presenting appropriate operation guide information in an eye operation.

### Solution to Problem

To attain the object described above, according to an embodiment of the present technique, there is provided a surgical image processing apparatus including circuitry configured to perform image recognition on an intraoperative image of an eye. The circuitry is further configured to determine a cross-section for acquiring a tomographic image based on a result of the image recognition.

With this structure, since the cross-section is determined based on the result of the image recognition of the intraoperative image, the user does not need to designate the cross-section. In addition, since the cross-section is determined according to a content of the intraoperative image (position and direction of eye and surgical instrument, etc.), the information processing apparatus can generate an appropriate tomographic image.

To attain the object described above, according to an embodiment of the present technique, there is provided an information processing method including performing, by circuitry of a surgical image processing apparatus, image recognition on an intraoperative image of an eye. The method further includes determining, by the circuitry, a cross-section for acquiring a tomographic image based on a result of the image recognition.

To attain the object described above, according to an embodiment of the present technique, there is provided a surgical microscope system including a surgical microscope and circuitry. The surgical microscope is configured to capture an image of an eye. The circuitry is configured to perform image recognition on an intraoperative image of an eye. The circuitry is configured to determine a cross-section for acquiring a tomographic image based on a result of the image recognition. The circuitry is further configured to control the surgical microscope to acquire the tomographic image of the cross-section.

### Effects of Invention

As described above, according to the present technique, it is possible to provide a surgical image processing apparatus, an information processing method, and a surgical microscope system that are capable of presenting appropriate operation guide information in an eye operation. It should be noted that the effects described herein are not necessarily limited and may be any effect described in the present disclosure.

### BRIEF DESCRIPTION OF DRAWINGS

FIG. 1 A block diagram showing a structure of an operation microscope apparatus according to an embodiment of the present technique.

FIG. 2 A block diagram showing a structure of an image information acquisition section of the operation microscope apparatus.

FIG. 3 A block diagram showing a structure of the image information acquisition section of the operation microscope apparatus.

FIG. 4 A block diagram showing a structure of the image information acquisition section of the operation microscope apparatus.

FIG. 5 A block diagram showing a structure of the image information acquisition section of the operation microscope apparatus.

FIG. 6 A block diagram showing a structure of the image information acquisition section of the operation microscope apparatus.

FIG. 7 A block diagram showing a structure of the image information acquisition section of the operation microscope apparatus.

FIG. 8 A block diagram showing a structure of the image information acquisition section of the operation microscope apparatus.

FIG. 9 A block diagram showing a structure of the image information acquisition section of the operation microscope apparatus.

FIG. 10 A block diagram showing a hardware structure of the operation microscope apparatus.

FIG. 11 A schematic diagram showing an operation process of a cataract operation in which the operation microscope apparatus can be used.

FIG. 12 A schematic diagram showing an operation process of the cataract operation in which the operation microscope apparatus can be used.

FIG. 13 A schematic diagram showing an operation process of the cataract operation in which the operation microscope apparatus can be used.

FIG. 14 A flowchart showing an operation of the operation microscope apparatus.

FIG. 15 An example of an intraoperative image acquired by the image information acquisition section of the operation microscope apparatus.

FIG. 16 A schematic diagram showing a cross-section determined by a controller of the operation microscope apparatus.

FIG. 17 An example of a tomographic image acquired by the image information acquisition section of the operation microscope apparatus.

FIG. 18 An example of guide information generated by a guide information generation section of the operation microscope apparatus.

FIG. 19 A schematic diagram showing a cross-section determined by the controller of the operation microscope apparatus.

FIG. 20 An example of the tomographic image acquired by the image information acquisition section of the operation microscope apparatus.

FIG. 21 An example of the tomographic image acquired by the image information acquisition section of the operation microscope apparatus.

FIG. 22 An example of a preoperative image acquired by the image information acquisition section of the operation microscope apparatus.

FIG. 23 A schematic diagram showing the cross-section determined by the controller of the operation microscope apparatus.

FIG. 24 An example of a preoperative tomographic image acquired by the image information acquisition section of the operation microscope apparatus.

FIG. 25 An example of the guide information generated by the guide information generation section of the operation microscope apparatus.

FIG. 26 An example of the guide information generated by the guide information generation section of the operation microscope apparatus.

FIG. 27 An example of the guide information generated by the guide information generation section of the operation microscope apparatus.

FIG. 28 An example of the guide information generated by the guide information generation section of the operation microscope apparatus.

FIG. 29 An example of the guide information generated by the guide information generation section of the operation microscope apparatus.

FIG. 30 An example of the guide information generated by the guide information generation section of the operation microscope apparatus.

FIG. 31 An example of the guide information generated by the guide information generation section of the operation microscope apparatus.

### DESCRIPTION OF EMBODIMENTS

Hereinafter, an operation microscope apparatus according to an embodiment of the present technique will be described.

[Structure of Operation Microscope Apparatus]

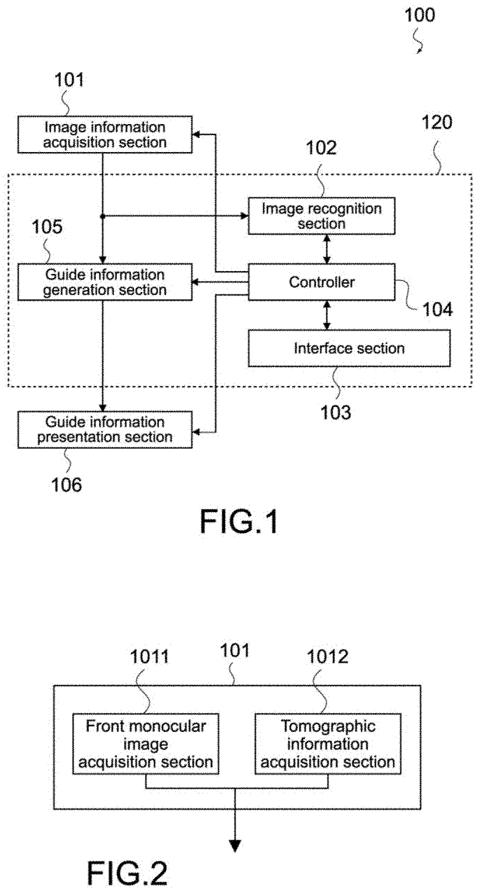

FIG. 1 is a block diagram showing a structure of an operation microscope apparatus 100 according to this embodiment. As shown in the figure, the operation microscope apparatus 100 includes an image information acquisition section 101, an image recognition section 102, an interface section 103, a controller 104, a guide information generation section 105, and a guide information presentation section 106. The image recognition section 102, the interface section 103, the controller 104, and the guide information generation section 105 are realized by an information processing apparatus 120.

The image information acquisition section 101 acquires image information of an operation target eye. The image information acquisition section 101 includes various structures with which image information such as a microscope image, a tomographic image, and volume data can be acquired. The various structures of the image information acquisition section 101 will be described later.

The image recognition section 102 executes image recognition processing on image information acquired by the image information acquisition section 101. Specifically, the image recognition section 102 recognizes an image of an surgical instrument or an eyeball site (pupil etc.) included in the image information. The image recognition processing may be executed by an edge detection method, a pattern matching method, and the like. The image recognition section 102 supplies the recognition result to the controller 104.

The interface section 103 acquires an image of an operation target eye taken before the operation, an operation plan, an instruction input from a user, and the like. The interface section 103 may also acquire a position or orientation of an surgical instrument measured by an optical position measurement apparatus. The interface section 103 supplies the acquired information to the controller 104.

The controller 104 determines a cross-section based on the recognition processing result obtained by the image recognition section 102. Specifically, the controller 104 can determine the cross-section based on the position or angle of the surgical instrument included in the image information, the eyeball site, and the like. The determination of the cross-section will be described later in detail.

The controller 104 also controls the image information acquisition section 101 to acquire a tomographic image of the determined cross-section. The controller 104 is also capable of controlling the respective structures of the operation microscope apparatus 100.

The guide information generation section 105 generates guide information for guiding an operation. The guide information is a tomographic image of a cross-section determined by the controller 104, an operation target line, a distance between the surgical instrument and the eyeball site, and the like. The guide information generation section 105 supplies the generated guide information to the guide information presentation section 106. The guide information generation section 105 generates an image including the guide information and supplies it to the guide information presentation section 106. The guide information generation section 105 may also generate the guide information as audio and supply it to the guide information presentation section 106.

The guide information presentation section 106 presents the guide information to the user. The guide information presentation section 106 is a display and is capable of displaying an image including the guide information generated by the guide information generation section 105. The guide information presentation section 106 is also a speaker and is capable of reproducing audio including the guide information generated by the guide information generation section 105.

[Regarding Image Information Acquisition Section]

The image information acquisition section 101 may include various structures. FIGS. 2 to 9 are block diagrams showing the various structures of the image information acquisition section 101.

As shown in FIG. 2, the image information acquisition section 101 may include a front monocular image acquisition section 1011 and a tomographic information acquisition section 1012. The front monocular image acquisition section 1011 may be a camera-equipped microscope or the like and is capable of taking a microscopic image of the operation target eye. The tomographic information acquisition section 1012 may be an OCT (Optical Coherence Tomography) or a shine-proof camera and is capable of taking a tomographic image of the operation target eye.

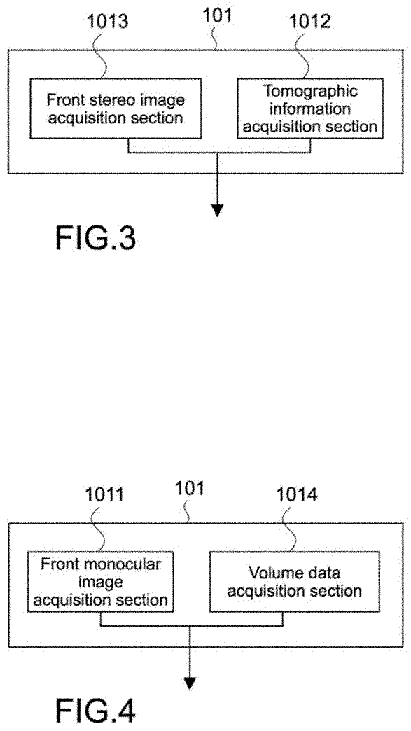

Further, as shown in FIG. 3, the image information acquisition section 101 may include a front stereo image acquisition section 1013 and the tomographic information acquisition section 1012. The front stereo image acquisition section 1013 may be a stereo camera-equipped microscope or the like and is capable of taking a microscopic stereo image of the operation target eye.

Furthermore, as shown in FIG. 4, the image information acquisition section 101 may include the front monocular image acquisition section 1011 and a volume data acquisition section 1014. The volume data acquisition section 1014 may be a tomographic image pickup mechanism such as the OCT and is capable of acquiring, by successively taking tomographic images, volume data (3D image) of the operation target eye.

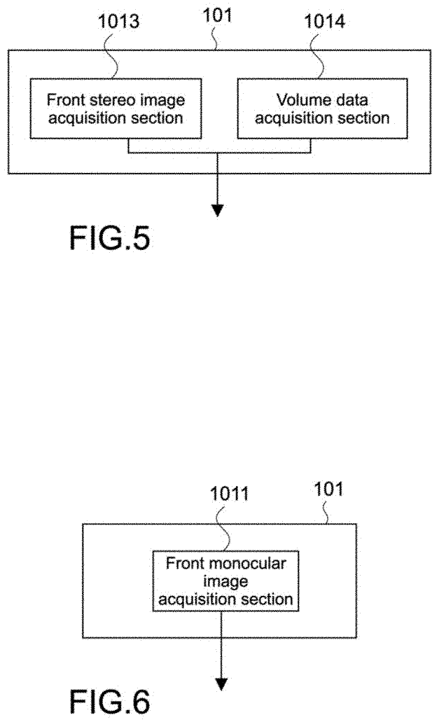

Moreover, as shown in FIG. 5, the image information acquisition section 101 may include the front stereo image acquisition section 1013 and the volume data acquisition section 1014.

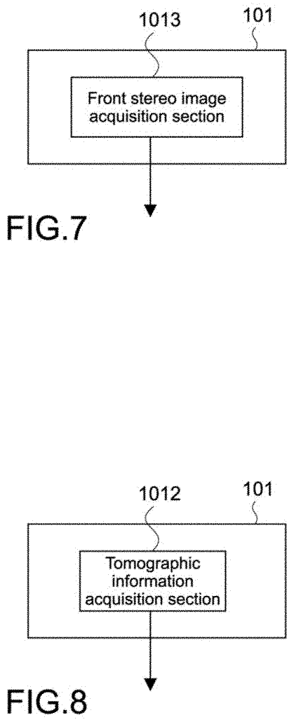

Further, the image information acquisition section 101 may be constituted of only the front monocular image acquisition section 1011 as shown in FIG. 6 or only the front stereo image acquisition section 1013 as shown in FIG. 7.

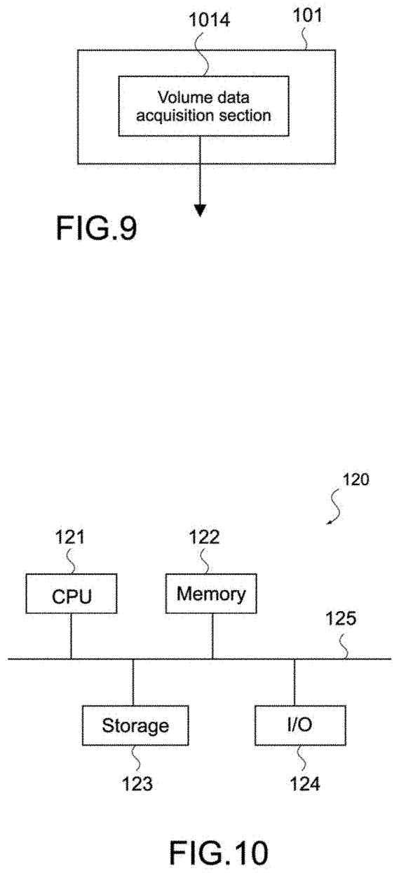

Furthermore, the image information acquisition section 101 may be constituted of only the tomographic information acquisition section 1012 as shown in FIG. 8 or only the volume data acquisition section 1014 as shown in FIG. 9.

[Hardware Structure]

The functional structure of the information processing apparatus 120 as described above can be realized by a hardware structure described below.

FIG. 10 is a schematic diagram showing the hardware structure of the information processing apparatus 120. As shown in the figure, the information processing apparatus 120 includes, as the hardware structure, a CPU 121, a memory 122, a storage 123, and an input/output section (I/O) 124, which are mutually connected by a bus 125.

The CPU (Central Processing Unit) 121 carries out, as well as control other structures according to a program stored in the memory 122, data processing according to a program and stores the processing result in the memory 122. The CPU 121 may be a microprocessor.

The memory 122 stores programs to be executed by the CPU 121 and data. The memory 122 may be a RAM (Random Access Memory).

The storage 123 stores programs and data. The storage 123 may be an HDD (Hard Disk Drive) or an SSD (Solid State Drive).

The input/output section 124 accepts an input to the information processing apparatus 120 and externally supplies an output of the information processing apparatus 120. The input/output section 124 includes an input apparatus such as a keyboard and a mouse, an output apparatus such as a display, and a connection interface for a network and the like.

The hardware structure of the information processing apparatus 120 is not limited to that described herein and only needs to be that capable of realizing the functional structure of the information processing apparatus 120. In addition, a part or all of the hardware structure may exist on a network.

[General Outline of Ophthalmic Operation]

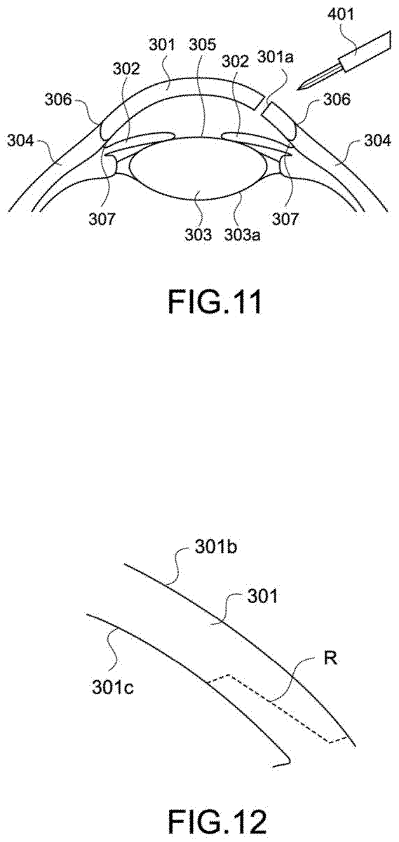

A generation outline of a cataract operation in which the operation microscope apparatus 100 can be used will be described. FIGS. 11 to 13 are schematic diagrams showing processes of the cataract operation. As shown in the figures, an eyeball is constituted of tissues of a cornea 301, an iris 302, a crystalline lens 303, a sclera 304, and the like. A pupil 305 is positioned inside the iris 302 on a surface of the crystalline lens 303, and an outer circumference of the cornea 301 is a corneal ring part 306. Angles 307 are positioned at both ends of the cornea 301.

As shown in FIG. 11, in the cataract operation, a incised wound 301a is formed on the cornea 301 by an surgical instrument 401 such as a knife. FIG. 12 is an enlarged view of the cornea 301 and shows an insertion path R of the surgical instrument 401. For closing the incised wound 301a after the operation, a method of inserting the surgical instrument 401 stepwise into the cornea 301 as shown in the figure so that the incised wound 301a is constituted of 3 incision surfaces is widely used. The insertion path R is determined based on a distance with respect to a corneal epithelium 301b on the surface of the cornea 301 or a corneal endothelium 301c on a back surface of the cornea 301.

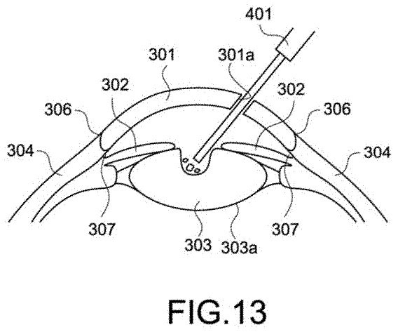

Next, as shown in FIG. 13, the surgical instrument 401 for aspiration is inserted from the incised wound 301a to aspirate and remove an inside (nucleus and cortical substance) of the crystalline lens 303. After that, an intraocular lens is inserted at a position from which the crystalline lens 303 has been removed, and the operation ends. In removing the crystalline lens 303, when the surgical instrument 402 is pressed by a posterior capsule 303a of the crystalline lens 303 or the posterior capsule 303a is aspirated to damage the posterior capsule 303a, an insertion of the intraocular lens becomes difficult. Therefore, there is a need to be careful so as not to damage the posterior capsule 303a.

It should be noted that the cataract operation described herein is an example of the ophthalmic operation in which the operation microscope apparatus 100 can be used, and the operation microscope apparatus 100 can be used in various ophthalmic operations.

[Operation of Operation Microscope Apparatus]

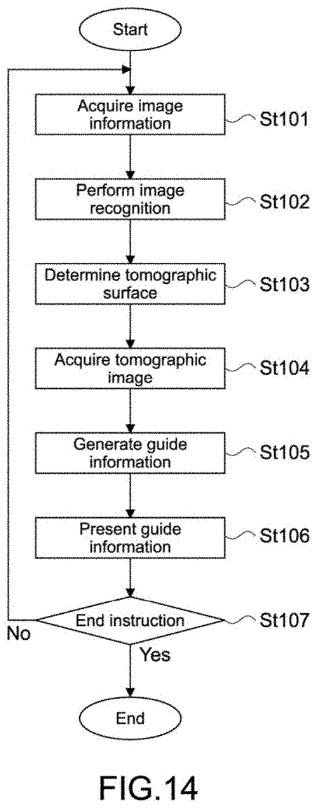

An operation of the operation microscope apparatus 100 will be described. FIG. 14 is a flowchart showing the operation of the operation microscope apparatus 100.

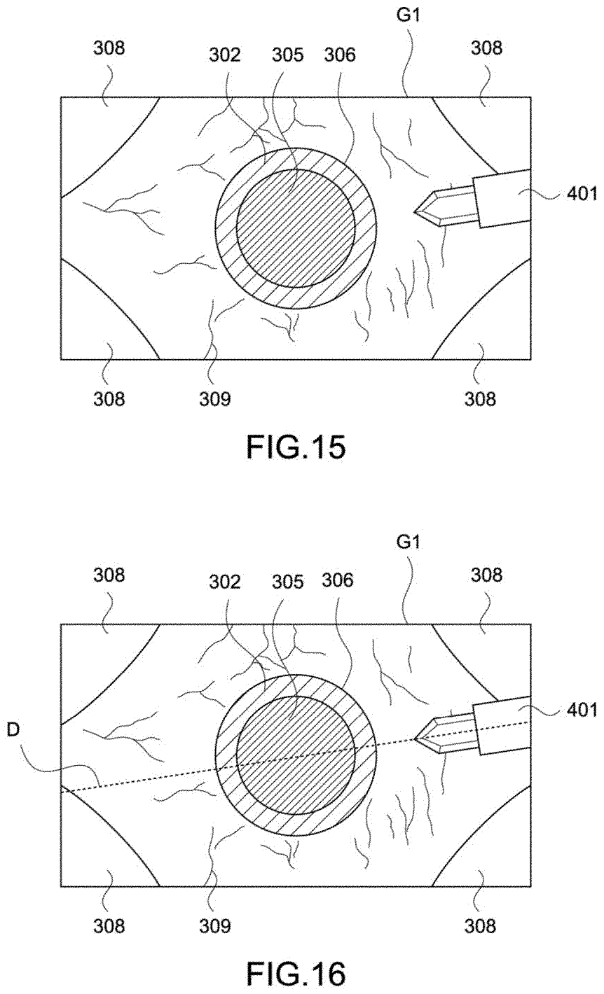

As a start instruction is input by a user, the controller 104 accepts the start instruction via the interface section 103 and starts processing. The controller 104 controls the image information acquisition section 101 to acquire image information of an operation target eye (St101). FIG. 15 is an example of an intraoperative image of the operation target eye acquired by the image information acquisition section 101. Hereinafter, this image will be referred to as intraoperative image G1. The intraoperative image G1 includes the surgical instrument 401, the pupil 305, the iris 302, an eyelid 308 opened by a lid retractor, and blood vessels 309. It should be noted that since the cornea 301 is transparent, an illustration thereof is omitted.

The image recognition section 102 executes image recognition processing on the intraoperative image G1 under control of the controller 104 (St102). The image recognition section 102 recognizes the surgical instrument 401 in the intraoperative image G1. The image recognition section 102 is capable of recognizing the surgical instrument 401 by comparing a preregistered pattern of the surgical instrument 401 and the intraoperative image G1, for example. At this time, the image recognition section 102 is capable of extracting a longitudinal direction of the surgical instrument 401 or positional coordinates thereof in the intraoperative image G1 as the image recognition result. The image recognition section 102 supplies the image recognition result to the controller 104.

Subsequently, the controller 104 determines a cross-section using the image recognition result (St103). FIG. 16 is a schematic diagram showing the cross-section determined by the controller 104. As shown in the figure, the controller 104 is capable of determining a surface D that passes a tip end position of the surgical instrument 401 and is parallel to the longitudinal direction of the surgical instrument 401 as the cross-section. It should be noted that although the surface D is expressed linearly in FIG. 16, the surface D is actually a surface that extends in a direction perpendicular to an image surface of the intraoperative image G1. The controller 104 is capable of determining the cross-section using other image recognition results, the descriptions of which will be given later.

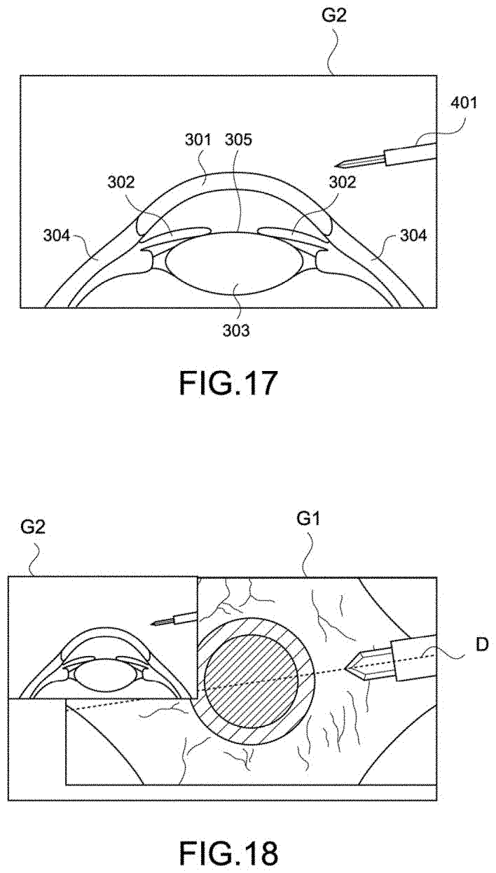

Next, the controller 104 controls the image information acquisition section 101 to acquire a tomographic image of an eye on the surface D (St104). FIG. 17 is an example of the tomographic image acquired by the image information acquisition section 101. Hereinafter, this image will be referred to as tomographic image G2. It should be noted that the controller 104 may acquire the tomographic image corresponding to the surface D from volume data acquired with respect to the operation target eye.

Subsequently, the guide information generation section 105 generates guide information. FIG. 18 is an example of the guide information. As shown in the figure, the guide information generation section 105 superimposes the intraoperative image G1 and the tomographic image G2 on top of each other to generate one image as the guide information. Alternatively, the guide information generation section 105 may use each of the intraoperative image G1 and the tomographic image G2 as the guide information. The guide information generation section 105 supplies the generated guide information to the guide information presentation section 106.

The guide information presentation section 106 presents the guide information supplied from the guide information generation section 105 to the user (St106). After that, the operation microscope apparatus 100 repetitively executes the steps described above until an end instruction is made by the user (St107: Yes). When the position or orientation of the surgical instrument 401 is changed by the user, the cross-section is determined according to that change, and a new tomographic image G2 is generated.

The operation microscope apparatus 100 performs the operation as described above. As described above, since a new tomographic image is presented according to the position or orientation of the surgical instrument 401, the user does not need to designate a desired cross-section.

[Regarding Other Cross-Section Determination Operations]

As described above, the controller 104 determines the cross-section based on the image recognition result obtained by the image recognition section 102. The controller 104 is also capable of determining the cross-section as follows.

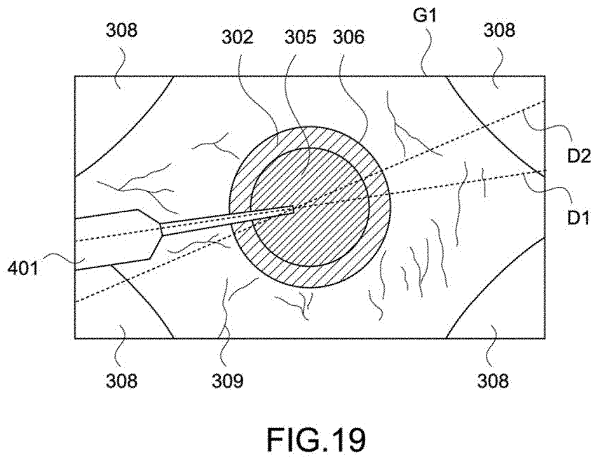

The controller 104 can determine a surface that passes the tip end position of the surgical instrument 401 recognized by the image recognition section 102 and is different from the longitudinal direction of the surgical instrument 401 as the cross-section. FIG. 19 is a schematic diagram of the intraoperative image G1 in this case. In the figure, the surface that passes the tip end position of the surgical instrument 401 and is parallel to the longitudinal direction of the surgical instrument 401 is a surface D1, and a surface that passes the tip end position of the surgical instrument 401 and forms a certain angle from the longitudinal direction of the surgical instrument 401 is a surface D2. The controller 104 can determine the surface D2 as the cross-section. An intersection angle of the surfaces D1 and D2 is arbitrary and may be orthogonal.

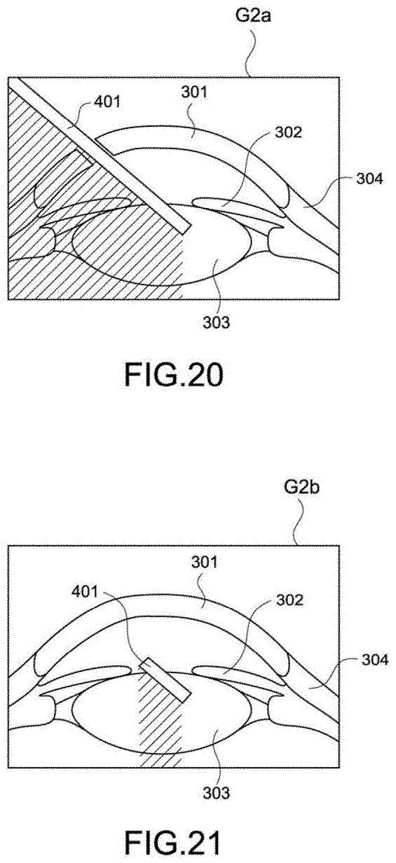

FIG. 20 shows a tomographic image G2a in a case where the surface D1 is the cross-section, and FIG. 21 shows a tomographic image G2b in a case where the surface D2 is the cross-section. As shown in FIG. 20, a tomographic image of an area shadowed by the surgical instrument 401 (hatched area) cannot be acquired favorably when the surface D1 is used as the cross-section. On the other hand, as shown in FIG. 21, the area shadowed by the surgical instrument 401 (hatched area) becomes small when the surface D2 is used as the cross-section, and it becomes easy to grasp the tomographic image. The area shadowed by the surgical instrument 401 is relatively large when the intersection angle of the surfaces D1 and D2 is small, but a similarity of a cross section of an eye that uses the surface D2 as the cross-section and a cross section of an eye that uses the surface D1 as the cross-section becomes high. Therefore, since the shadowed area is reduced as compared to the tomographic image that uses the surface D1 as the cross-section, it becomes that much easier to grasp a state of the operation target site in the tomographic image that uses the surface D2 as the cross-section. On the other hand, when the surfaces D1 and D2 are orthogonal to each other, the area shadowed by the surgical instrument 401 becomes minimum. The controller 104 may determine either the surface D1 or D2 as the cross-section or both the surfaces D1 and D2 as the cross-sections.

The guide information generation section 105 is capable of generating guide information including one of or both the tomographic image G2a and the tomographic image G2b. It should be noted that the controller 104 may determine 3 or more surfaces as the cross-sections and cause tomographic images of the cross-sections to be acquired.

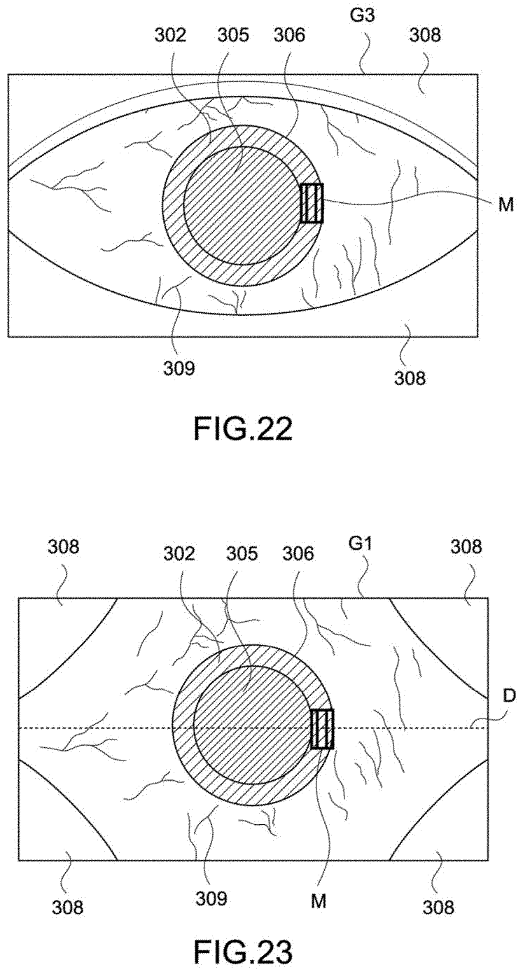

The controller 104 is also capable of determining the cross-section based on the incised wound creation position designated in the preoperative plan. FIG. 22 is an example of a preoperative image that has been taken preoperatively. Hereinafter, this image will be referred to as preoperative image G3. The user can designate a incised wound creation position M in the preoperative image G3. The incised wound creation position M is a position at which the incised wound 301a is formed in the incised wound creation process (see FIG. 11). As shown in FIG. 22, the incised wound creation position M can be expressed by a projection view of 3 surfaces for expressing 3 incision surfaces that are the same as the insertion path R shown in FIG. 12.

The controller 104 acquires the preoperative image G3 in which the incised wound creation position M is designated from the image information acquisition section 101 or the interface section 103 and supplies it to the image recognition section 102 at a stage before the operation start. When the operation is started and the intraoperative image G1 is taken, the image recognition section 102 compares the intraoperative image G1 and the preoperative image G3. The image recognition section 102 is capable of detecting, by comparing locations of the eyeball sites (e.g., blood vessels 309) included in the images, a difference in the positions or angles of the eye in the images. The image recognition section 102 supplies the difference to the controller 104.

The controller 104 specifies the incised wound creation position M in the intraoperative image G1 based on the difference between the intraoperative image G1 and the preoperative image G3 detected by the image recognition section 102. FIG. 23 is a schematic diagram showing the incised wound creation position M specified in the intraoperative image G1. The controller 104 is capable of determining the surface that passes the incised wound creation position M as the cross-section. For example, the controller 104 is capable of determining a surface D that passes a center of the incised wound creation position M and the pupil 305 as the cross-section as shown in FIG. 23. Moreover, the controller 104 may determine a surface that passes other eyeball sites and the incised wound creation position M, such as a center of the corneal ring part 306, as the cross-section.

It should be noted that the user may designate a cross-section for which the user wishes to reference a tomographic image instead of the incised wound creation position M in the preoperative image G3. The controller 104 is also capable of specifying in the intraoperative image G1, based on the difference between the intraoperative image G1 and the preoperative image G3 as described above, a surface corresponding to the cross-section designated in the preoperative image G3 and determining it as the cross-section.

[Regarding Other Guide Information Generation Operations]

As described above, the guide information generation section 105 is capable of generating guide information including a front image and a tomographic image. The guide information generation section 105 may also generate the guide information as follows.

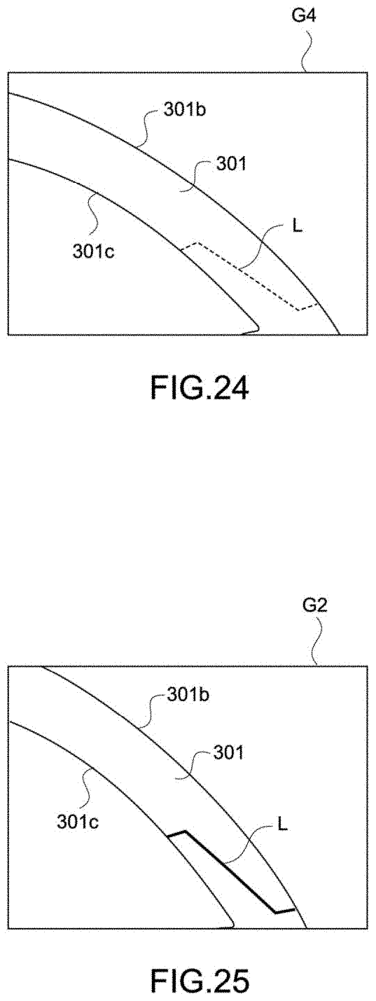

The guide information generation section 105 can generate the guide information by superimposing a target line on the tomographic image acquired as described above. The user can designate an arbitrary cross-section in the preoperative image G3, and the controller 104 controls the image information acquisition section 101 to acquire a tomographic image of the designated cross-section. FIG. 24 is a schematic diagram of the tomographic image acquired preoperatively (hereinafter, referred to as tomographic image G4). As shown in the figure, the user can preoperatively designate a target line L while referencing the eyeball site (corneal epithelium 301b, corneal endothelium 301c, etc.) in the tomographic image G4.

As described above, upon start of the operation, the controller 104 compares the intraoperative image G1 and the preoperative image G3 and determines a surface to be a cross-section based on a difference between the images (see FIG. 23). The controller 104 controls the image information acquisition section 101 to acquire the tomographic image G2 of the determined cross-section. The guide information generation section 105 compares the tomographic image G4 and the tomographic image G2 and detects a difference between the images. The difference between the images can be detected using two or more feature points (e.g., angles 307) in the tomographic image.

FIG. 25 is an example of the guide information including the tomographic image G2. As shown in the figure, the guide information generation section 105 is capable of generating, based on the difference between the images, the guide information in which the target line L is arranged in the tomographic image G2 so as to coincide with the positional relationship of the target line L designated in the tomographic image G4. Accordingly, during the operation, the user can reference the target line L set in the preoperative plan in the tomographic image of the same cross-section as the preoperative plan.

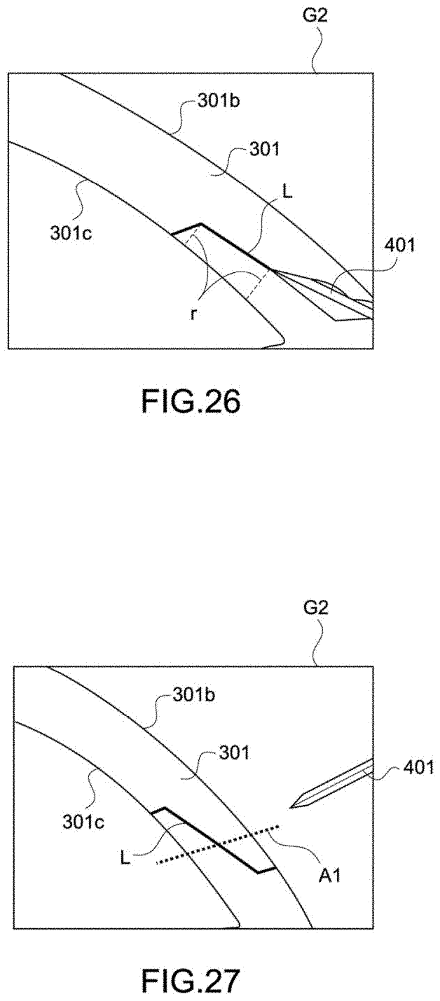

Further, the guide information generation section 105 may dynamically change the target line L along with a progress of the operation. FIG. 26 is a schematic diagram of the guide information including the tomographic image G2 in the incised wound creation process (see FIG. 11). In the figure, the incision of the cornea 301 by the surgical instrument 401 is partway done. The guide information generation section 105 is capable of deforming the target line L such that a distance between the target line L and the corneal endothelium 301c (r in figure) becomes the same as that of the preoperative plan.

Furthermore, the guide information generation section 105 may deform the target line L using a distance between the target line L and the corneal epithelium 301b as a reference. In addition, the guide information generation section 105 is capable of deleting the target line L for an incised part. As a result, it becomes possible to display the target line L while reflecting a deformation of the cornea due to the incision.

Further, the guide information generation section 105 may generate guide information including angle information. FIG. 27 is a schematic diagram of the guide information including the tomographic image G2. In the tomographic image G2, a target angle A1 is indicated. The guide information generation section 105 can set an angle of the target line L at the tip end position of the surgical instrument as the target angle in the tomographic image G2. In FIG. 27, since the surgical instrument 401 is not inserted into the cornea 301, the target angle A1 is an angle of the target line L at an insertion start side end part.

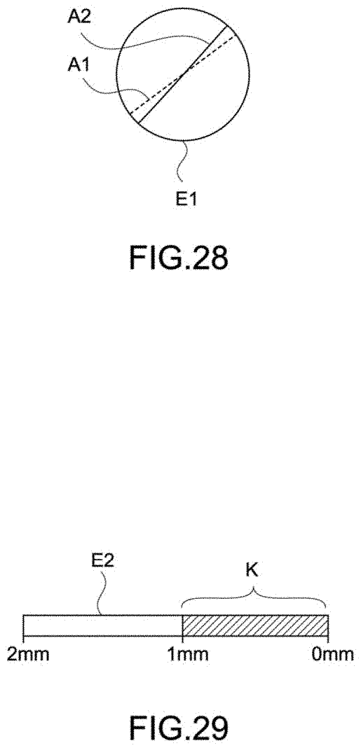

The guide information generation section 105 may generate an indicator that expresses the angle information. FIG. 28 is an example of an angle indicator E1 indicating the angle information. In the angle indicator E1, a broken line indicates the target angle A1, and a solid line indicates an actual angle A2 as the angle of the surgical instrument 401. The guide information generation section 105 acquires the angle of the surgical instrument 401 measured (recognized) by the image recognition section 102 via the controller 104. The image recognition section 102 may acquire the angle of the surgical instrument 401 by the image recognition with respect to the tomographic image G2, acquire the angle by the image recognition with respect to a front stereo image taken by the front stereo image acquisition section 1013, or acquire the angle of the surgical instrument 401 measured by an optical position measurement apparatus from the interface section 103. It should be noted that regarding the target angle A1 in the indicator E1, an arbitrary fixed angle in a horizontal direction or the like may be used instead of using the angle of the target line L in the tomographic image G2 as it is. In this case, a relative angle of the target angle and the surgical instrument angle in the indicator can be made to coincide with that of the measured (recognized) target angle and surgical instrument angle.

Moreover, the guide information generation section 105 may generate guide information including distance information on the tip end of the surgical instrument 401 and the eyeball site. FIG. 29 is an example of a distance indicator E2 indicating the distance information. In the distance indicator E2, a distance K indicates a distance between the surgical instrument tip end and the eyeball site and extends/contracts according to the actual distance. The guide information generation section 105 acquires the distance measured (recognized) by the image recognition section 102 via the controller 104. The image recognition section 102 is capable of acquiring the distance between the surgical instrument tip end and the eyeball site by the image recognition with respect to the tomographic image G2. The image recognition section 102 can also acquire the distance based on the front stereo image taken by the front stereo image acquisition section 1013.

Further, the image recognition section 102 may estimate a distribution of the eyeball site from the comparison between a feature point in the preoperative tomographic image G4 or volume data and a feature point in the intraoperative tomographic image G2 or volume data and estimate the distance between the surgical instrument tip end and the eyeball site. The image recognition section 102 may also acquire the position of the surgical instrument tip end based on the position or orientation of the surgical instrument 401 measured by the optical position measurement apparatus and estimate the distance between the surgical instrument tip end and the eyeball site based on the positional relationship with the feature points of the front stereo image and the like.

It should be noted that the feature points can be set as the position of the corneal ring part 306 in the tomographic image, apexes of the corneal ring part 306 and the cornea 301 in the volume data, and the like.

The eyeball site for which the distance with respect to the surgical instrument tip end is to be acquired is not particularly limited but is favorably the posterior capsule 303a, the corneal endothelium 301c, an eyeball surface, or the like. The distance between the surgical instrument tip end and the posterior capsule 303a is effective for preventing the posterior capsule 303a from being damaged by the aspiration process (see FIG. 13) of the crystalline lens, and the distance between the surgical instrument tip end and the corneal endothelium 301c is effective for grasping the distance between the surgical instrument tip end and the corneal endothelium 301c in the aspiration process of the crystalline lens or at the time of adjusting the position of the intraocular lens. In addition, the distance between the surgical instrument tip end and the eyeball surface is effective for grasping the distance between the eyeball surface and the surgical instrument tip end in the incised wound creation process (see FIG. 11).

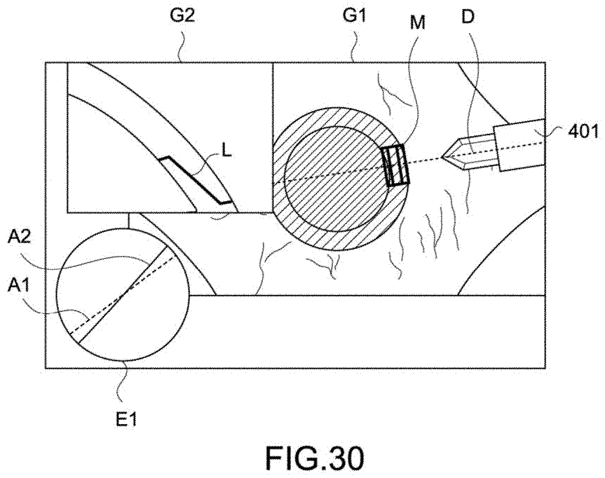

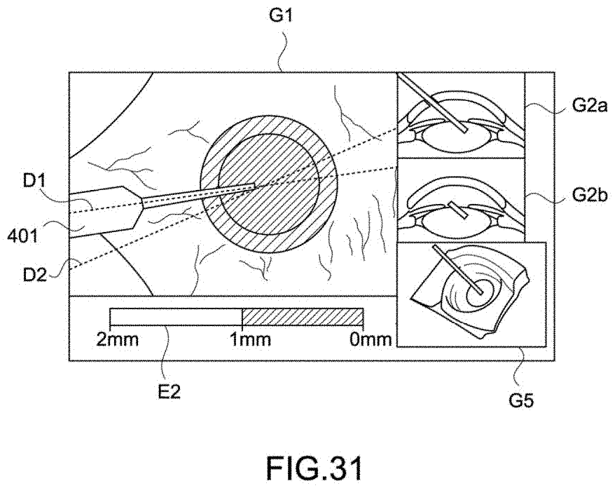

FIGS. 30 and 31 are examples of the guide information generated by the guide information generation section 105. As shown in FIG. 30, the guide information may include the intraoperative image G1, the tomographic image G2 including the target line L, the angle indicator E1, the incised wound creation position M, and the surface D for which the tomographic image G2 has been acquired. Moreover, as shown in FIG. 31, the guide information may include the tomographic image G2a, the tomographic image G2b, the surface D1 for which the tomographic image G2a has been acquired, the surface D2 for which the tomographic image G2b has been acquired, the distance indicator E2, and the volume data G5. The guide information may include any of those described above.

It should be noted that the guide information generation section 105 may generate audio instead of an image as the guide information. Specifically, the guide information generation section 105 may use as the guide information an alarm sound obtained by varying a frequency or volume according to the distance between the surgical instrument tip end and the eyeball site described above.

Further, the guide information generation section 105 can also use as the guide information an alarm sound whose volume is varied according to the deviation amount from the target line, like a high frequency is set when the surgical instrument is facing upward higher than the target line L (see FIG. 28) and a low frequency is set when the surgical instrument is facing downward lower than the target line.

It should be noted that the present technique may also take the following structures.

(1)

A surgical image processing apparatus, including:

circuitry configured to

perform image recognition on an intraoperative image of an eye; and

determine a cross-section for acquiring a tomographic image based on a result of the image recognition.

(2)

The surgical image processing apparatus according to (1), in which the circuitry is configured to

recognize an image of a surgical instrument in the intraoperative image, and determine the cross-section based on the image of the surgical instrument.

(3)

The surgical image processing apparatus according to (2), in which the cross-section passes a position of a tip end of the surgical instrument.

(4)

The surgical image processing apparatus according to (2) or (3), in which the circuitry is configured to

determine the cross-section based on a longitudinal direction of the surgical instrument.

(5)

The surgical image processing apparatus according to any one of (2) to (4),

in which the cross-section passes a position of a tip end of the surgical instrument and is parallel or at a predetermined angle to a longitudinal direction of the surgical instrument.

(6)

The surgical image processing apparatus according to any one of (1) to (5), in which the circuitry is configured to

compare a preoperative image of the eye with the intraoperative image of the eye, and

determine the cross-section based on a result of the comparison.

(7)

The surgical image processing apparatus according to (6), in which the circuitry is configured to

specify, based on the result of the comparison, an incised wound creation position in the intraoperative image, that has been designated in the preoperative image, and determine the cross-section based on the incised wound creation position in the intraoperative image.

(8)

The surgical image processing apparatus according to (7),

in which the cross-section passes through the incised wound creation position in the intraoperative image.

(9)

The surgical image processing apparatus according to (7) or (8), in which the circuitry is configured to

recognize a feature of the eye in the intraoperative image, and determine the cross-section based on the incised wound creation position and the feature of the eye in the intraoperative image.

(10)

The surgical image processing apparatus according to (9),

in which the feature of the eye is a pupil, iris, eyelid, or blood vessel of the eye.

(11)

The surgical image processing apparatus according to any one of (1) to (10), in which the circuitry is configured to

control an image sensor that acquires image information of the eye to acquire the tomographic image of the cross-section.

(12)

The surgical image processing apparatus according to any one of (1) to (11), in which the circuitry is configured to

generate guide information for an operation based on the tomographic image of the cross-section.

(13)

The surgical image processing apparatus according to (12),

in which the guide information includes at least one of the tomographic image of the cross-section, operation target position information, or distance information regarding a surgical instrument and a feature of the eye.

(14)

The surgical image processing apparatus according to (13),

in which the distance information indicates the distance between the surgical instrument and the feature of the eye.

(15)

The surgical image processing apparatus according to (13) or (14),

in which the feature of the eye is a posterior capsule of the eye.

(16)

The surgical image processing apparatus according to any one of (12) to (15),

in which the guide information includes distance information that indicates distances between a surgical instrument and a plurality of features of the eye.

(17)

The surgical image processing apparatus according to any one of (13) to (16),

in which the distance information is calculated based on a plurality of images of the eye captured by a stereo camera.

(18)

The surgical image processing apparatus according to any one of (13) to (17), in which the circuitry is configured to

control an image sensor that acquires image information of the eye to acquire a preoperative tomographic image of the eye and an intraoperative tomographic image of the eye corresponding to the cross-section, and

generate the operation target position information in the intraoperative tomographic image based on a preoperatively designated position in the preoperative tomographic image.

(19)

The surgical image processing apparatus according to any one of (13) to (18), further including

at least one of a display or a speaker configured to present an image or audio corresponding to the guide information generated by the circuitry to a user.

(20)

The surgical image processing apparatus according to any one of (1) to (19), in which the circuitry is configured to

dynamically change the cross-section according to changes in a position or orientation of a surgical instrument.

(21)

The surgical image processing apparatus according to any one of (1) to (20), in which the circuitry is configured to

concurrently display a preoperative tomographic image and an intraoperative tomographic image of the eye.

(22)

An surgical image processing method, including:

performing, by circuitry of an image processing apparatus, image recognition on an intraoperative image of an eye; and

determining, by the circuitry, a cross-section for acquiring a tomographic image based on a result of the image recognition.

(23)

A surgical microscope system, including:

a surgical microscope configured to capture an image of an eye; and circuitry configured to
perform image recognition on an intraoperative image of an eye,
determine a cross-section for acquiring a tomographic image based on a result of the image recognition, and
control the surgical microscope to acquire the tomographic image of the cross-section.

(24)

The surgical microscope system according to (23),

in which the surgical microscope is configured to capture a stereoscopic image.

### REFERENCE SIGNS LIST

100 operation microscope apparatus
101 image information acquisition section
102 image recognition section
103 interface section
104 controller
105 guide information generation section
106 guide information presentation section

1. A surgical image processing apparatus, comprising:
circuitry configured to
perform image recognition on an intraoperative image of an eye;
determine a cross-section for acquiring a tomographic image based on a result of the image recognition; and
generate guide information for an operation based on the tomographic image of the cross-section.

2. The surgical image processing apparatus according to claim 1, wherein the guide information includes at least one of the tomographic image of the cross-section, operation target position information, or distance information regarding a surgical instrument and a feature of the eye.

3. The surgical image processing apparatus according to claim 2, wherein the distance information indicates the distance between the surgical instrument and the feature of the eye.

4. The surgical image processing apparatus according to claim 2, wherein the feature of the eye is a posterior capsule of the eye.

5. The surgical image processing apparatus according to claim 1, wherein the guide information includes distance information that indicates distances between a surgical instrument and a plurality of features of the eye.

6. The surgical image processing apparatus according to claim 2, wherein the distance information is calculated based on a plurality of images of the eye captured by a stereo camera.

7. The surgical image processing apparatus according to claim 2, wherein the circuitry is further configured to:
control an image sensor that acquires image information of the eye to acquire a preoperative tomographic image of the eye and an intraoperative tomographic image of the eye corresponding to the cross-section; and
generate the operation target position information in the intraoperative tomographic image based on a preoperatively designated position in the preoperative tomographic image.

8. The surgical image processing apparatus according to claim 2, further comprising at least one of a display or a speaker configured to present an image or audio corresponding to the guide information generated by the circuitry to a user.

9. The surgical image processing apparatus according to claim 1, wherein the circuitry is further configured to concurrently display a preoperative tomographic image and an intraoperative tomographic image of the eye.

10. An information processing method, comprising:
performing, by circuitry of a surgical image processing apparatus, image recognition on an intraoperative image of an eye;
determining, by the circuitry, a cross-section for acquiring a tomographic image based on a result of the image recognition; and
generating guide information for an operation based on the tomographic image of the cross-section.

11. The information processing method according to claim 10, wherein the guide information includes at least one of the tomographic image of the cross-section, operation target position information, or distance information regarding a surgical instrument and a feature of the eye.

12. The information processing method according to claim 11, wherein the distance information indicates the distance between the surgical instrument and the feature of the eye.

13. The information processing method according to claim 11, wherein the feature of the eye is a posterior capsule of the eye.

14. The information processing method according to claim 10, wherein the guide information includes distance information that indicates distances between a surgical instrument and a plurality of features of the eye.

15. The information processing method according to claim 11, wherein the distance information is calculated based on a plurality of images of the eye captured by a stereo camera.

16. The information processing method according to claim 11, further comprising:
controlling an image sensor that acquires image information of the eye to acquire a preoperative tomographic image of the eye and an intraoperative tomographic image of the eye corresponding to the cross-section; and
generating the operation target position information in the intraoperative tomographic image based on a preoperatively designated position in the preoperative tomographic image.

17. The information processing method according to claim 11, further comprising presenting an image or audio corresponding to the guide information from at least one of a display or a speaker to a user.

18. The information processing method according to claim 10, further comprising concurrently displaying a preoperative tomographic image and an intraoperative tomographic image of the eye.

19. A surgical microscope system, comprising:
a surgical microscope configured to capture an image of an eye; and
circuitry configured to:
perform image recognition on an intraoperative image of an eye;
determine a cross-section for acquiring a tomographic image based on a result of the image recognition;
control the surgical microscope to acquire the tomographic image of the cross-section; and
generate guide information for an operation based on the tomographic image of the cross-section.

20. The surgical microscope system according to claim 19, wherein the surgical microscope is configured to capture a stereoscopic image.
